(19) United States
(12) Patent Application Publication
(10) Pub. No.: US 2024/0407939 A1
(43) Pub. Date: Dec. 12, 2024

(54) BAND FOR PREVENTING CONTACT OF INJURY

(71) Applicant: Andrew LEE, Portland, OR (US)

(72) Inventor: Andrew LEE, Portland, OR (US)

(21) Appl. No.: 18/796,194

(22) Filed: Aug. 6, 2024

(51) Int. Cl. A61F 5/30

(52) U.S. Cl. CPC A61F 5/30

(57) ABSTRACT

An injury contact prevention band includes an injury cover portion, a fastening portion, and a hole cover portion. The injury cover portion covers a part of a body including an injury and has a thickness through which an injury opening hole cuts to expose the injury. The fastening portion fastens the injury cover portion to the body. The hole cover portion covers the injury opening hole and is separatable from the injury opening hole. The fastening portion includes a first fastening member extending from an end of the injury cover portion and having flexibility, a second fastening member extending from another end of the injury cover portion and having flexibility, and a connecting member detachably connecting the end of the first fastening member and the end of the second fastening member to each other. The hole cover portion includes a first cover member and a second cover member.

### BACKGROUND

The present disclosure relates to a band, and more particularly to a band that prevents contact of a patient's injury.

In general, in the case of patients who lie in bed for a long period of time due to difficulty in movement, or paralyzed patients such as paralysis of the lower body or the entire body, bedsores easily form in areas that are under pressure because of poor blood circulation and metabolism due to remaining in the same posture for a long period of time.

In order to prevent these bedsores, the patient's lying position is often changed at regular intervals to relieve the burden on the pressured area. For this purpose, it is effective for the patient to lie down on his or her side facing the left or right at regular intervals to relieve the pressure on the back and buttocks, where bedsores usually occur, while the patient is lying down.

Additionally, if an injury such as a bedsore has already occurred, if the injury comes into contact with a bed mattress or blanket, pain may be caused, and injury recovery may be delayed. Even if the bedsore area comes into contact with a flexible and soft object, such as a bandage used for general injuries, it may cause pain.

To prevent such contact, bedding that supports a lying patient has been developed. However, such bedding generally has a large volume, so it is difficult to use in small houses and is difficult or bulky to carry. In addition, although it is possible to prevent contact with the back area, contacts with other areas may not be prevented.

### SUMMARY

An injury contact prevention band includes an injury cover portion, a fastening portion, and a hole cover portion. The injury cover portion covers a part of a body including an injury and has a thickness through which an injury opening hole cuts to expose the injury. The fastening portion fastens the injury cover portion to the body. The hole cover portion covers the injury opening hole, and is separatable from the injury opening hole. The fastening portion includes a first fastening member extending from an end of the injury cover portion and having flexibility, a second fastening member extending from another end of the injury cover portion and having flexibility, and a connecting member detachably connecting the end of the first fastening member and the end of the second fastening member to each other. The hole cover portion includes a first cover member and a second cover member. The outer perimeter surface fittingly engages with the inner perimeter surface of the injury opening hole. A first open hole is located in the first cover member. The second cover member covers the first open hole. The outer perimeter surface fittingly engages with the inner perimeter surface of the first open hole.

### BRIEF DESCRIPTION OF DRAWINGS

The above and other aspects, features, and advantages of the present disclosure will be more clearly understood from the following detailed description, taken in conjunction with the accompanying drawings.

FIG. 1 is a perspective view illustrating an injury contact prevention band according to an example embodiment of the present disclosure.

FIG. 2 is a perspective view illustrating an example of the injury contact prevention band of FIG. 1 being separated.

FIG. 3 is a side cross-sectional view of the area where the injury opening hole of the injury cover portion illustrated in FIG. 1 is formed, cut from the inner side to the outer side.

FIG. 4 is a front view illustrating an example of a fastening portion to which an extension member is connected.

FIG. 5 is an enlarged front view of the injury opening hole illustrated in FIG. 1 with the hole cover portion connected.

FIG. 6 is a front view illustrating the hole cover portion and the injury opening hole in a state in which the second cover member illustrated in FIG. 1 is separated.

FIG. 7 is a front view illustrating a hole cover portion according to an example embodiment.

FIG. 8 is a perspective view illustrating an example of the hard cap portion.

FIG. 9 is a side cross-sectional view illustrating the hard cap portion of FIG. 8 inserted into the injury opening hole.

FIG. 10 is a partial front view illustrating an injury contact prevention band according to an example embodiment.

FIG. 11 is a partial front view illustrating an injury contact prevention band according to an example embodiment.

### DETAILED DESCRIPTION

Hereinafter, embodiments of the present disclosure will be described in detail and clearly to such an extent that an ordinary one in the art easily implements the inventive concept.

In this specification, the inside refers to the surface or area facing the body part on which the band may be worn on the body, and the outside refers to the surface or area facing away from the body.

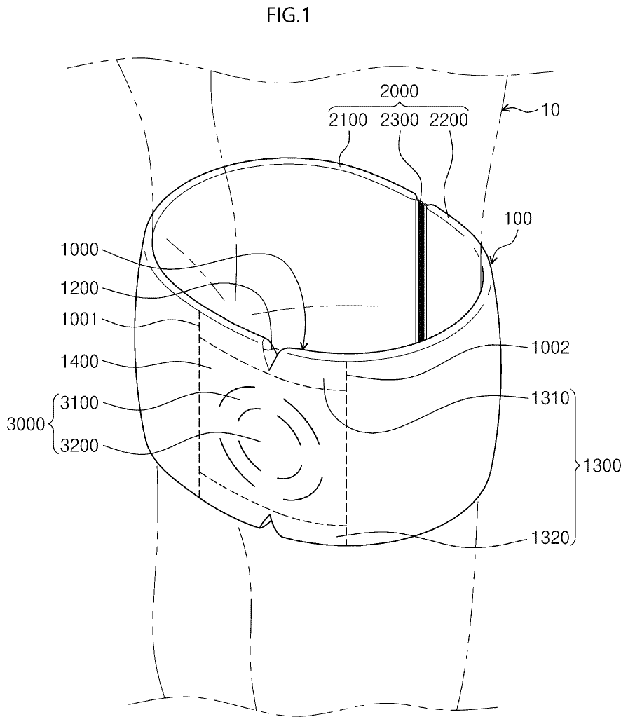

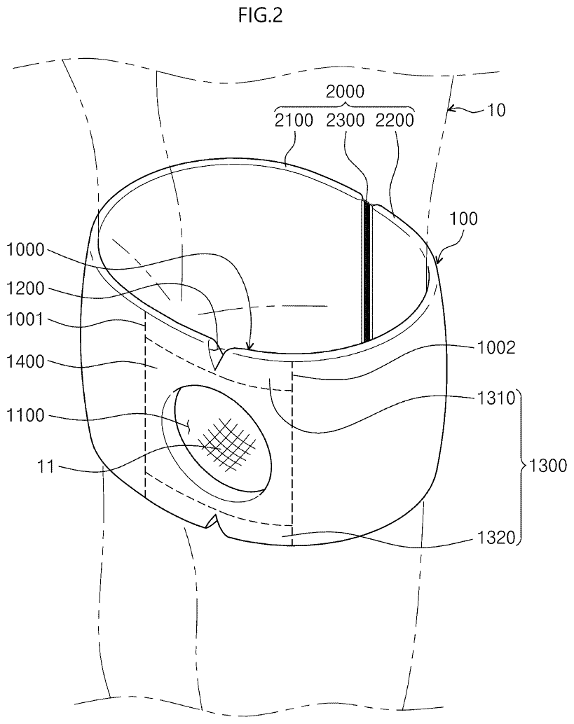

FIG. 1 is a perspective view illustrating an injury contact prevention band 100 according to an embodiment. FIG. 2 is a perspective view illustrating an example of the injury contact prevention band 100 of FIG. 1 being separated. The injury contact prevention band 100 prevents an injury or wound 11 from contacting an external object while fastened to a patient's body 10. For example, external objects may include bedding such as blankets or bed mattresses. In FIG. 1, the injury contact prevention band 100 is illustrated fastened to the pelvis portion so that the injury cover portion 1000 is positioned in a position covering one side of the buttocks. However, unlike this, the injury contact prevention band 100 may be fastened to various parts of the body 10 such as the arm or thigh so that an injury cover portion 1000 may cover the area where the injury 11 occurs. For example, an injury or a wound may be a variety of injuries that may be painful or infected upon contact, such as bedsores, surgical sites, or skin problems. The injury contact prevention band 100 may include an injury cover portion 1000, a fastening portion 2000, and a hole cover portion 3000.

The injury cover portion 1000 may cover a part of the body 10 including the injury 11. The injury cover portion 1000 may have flexibility and elasticity. For example, the injury cover portion 1000 may be made of a natural or synthetic material having flexibility and/or elasticity, such as leather, rubber, polyurethane, or acrylic resin. In another embodiment, the injury cover portion 1000 may have a structure in which components of different materials are combined. For example, the injury cover portion 1000 may be provided in a structure in which fabrics of different materials overlap each other to form a plurality of layers.

In one embodiment, the injury cover portion 1000 has an injury opening hole 1100 and a cut portion 1200.

The injury opening hole 1100 cuts through the entire thickness of injury cover portion 1000, or penetrates between an inner surface and an outer surface of the injury cover portion 1000 to expose, reveal, show, or display the injury 11 of the fastened body 10 to the outside. In one embodiment, the injury opening hole 1100 may be located singularly in the center of the injury cover portion 1000. The injury opening hole 1100 may have circular, square, rectangular, triangular, oval, elliptical, or any suitable shape.

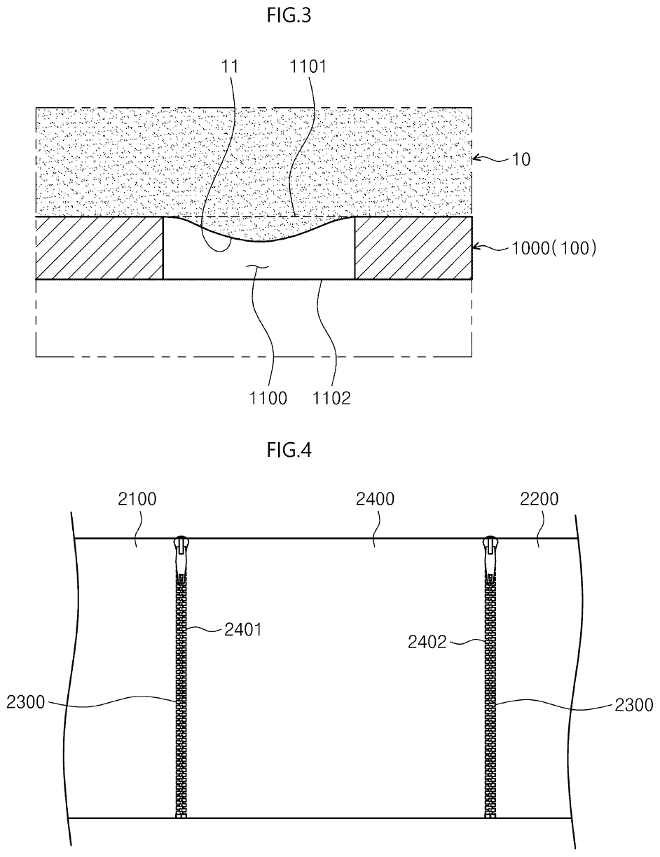

FIG. 3 is a side cross-sectional view of the area where the injury opening hole 1100 of the injury cover portion 1000 illustrated in FIG. 1 is positioned, cut from the inner side to the outer side. Referring to FIGS. 1 to 3, the injury contact prevention band 100 is fastened to the body 10 so that the open injury opening hole 1100 is positioned facing the injury 11 site. The injury cover portion 1000 may be provided with a thickness greater than the protrusion height of the injury 11 depending on the protrusion height of the injury 11 from the surrounding skin. The thickness of the injury cover portion 1000 may be provided by considering the degree to which the injury cover portion 1000 is compressed by the weight of the patient wearing it. However, the thicker the injury cover portion 1000, the greater the burden on the patient, such as making it difficult to move the worn part when worn, so the thickness of the injury cover portion 1000 relative to the protrusion height of the injury may be determined by test wearing or by other experiments or simulations, by considering the above-mentioned conditions. Therefore, the injury that opens through the injury opening hole 1100 is prevented from being contacted by the injury cover portion 1000, and prevented from contact with external objects by being surrounded by the injury opening hole 1100. The operating principle and effect of the first opening hole 3110 in FIG. 5, which will be described below, is also the same or similar to the above-described operating principle and effect of the injury opening hole 1100.

The injury opening hole 1100 may have various shapes such as square or oval. For example, the injury opening hole 1100 may be provided in a slit shape having an elongated shape in one direction. In this way, the injury opening hole 1100 are provided in various shapes, so that the user may selectively use the injury contact prevention band 100 having the injury opening hole 1100 suitable for the shape and size of the injury.

When a plurality of the injury opening holes 1100 are provided, one injury contact prevention band 100 may have injury opening holes 1100 of different shapes.

Referring again to FIGS. 1 and 2, the cut portion 1200 is formed in the end area 1300 of the injury cover portion 1000.

The end area 1300 is between the end and the other end of an edge area of the injury cover portion 1000. In one embodiment, the end area 1300 includes a first edge area 1310 and a second edge area 1320.

The first edge area 1310 is one of the areas between the end 1001 and the other end 1002 of the edge area of the injury cover portion 1000. The second edge area 1320 is another one of the areas between the end 1001 and the other end 1002 of the edge area of the injury cover portion 1000.

In one embodiment, the cut portion 1200 may be located in the middle between the end 1001 and the other end 1002 of the injury cover portion 1000 in each of the first edge area 1310 and the second edge area 1320. The cut portion 1200 is formed by cutting into a recessed shape from the outer perimeter the injury cover portion 1000 toward the central area 1400. The cut portion 1200 may be provided in a wedge shape that becomes wider as it moves away from the central area 1400.

As described above, the cut portion 1200 is located in the end area 1300, so that when the injury contact prevention band 100 is fastened to the body 10, each part cut by the cut portion 1200 in the end area may move more freely and flexibly inward and outward according to the curve of the body 10, thereby the lifting part from the skin caused by mutual interference may be reduced. The farther the part of the injury cover portion 1000 is from the central area 1400, the more movement may occur relative to the skin when the body 10 moves, and the part that is lifted from the skin may have more movement compared to the part that is not lifted. The part that is lifted from the skin causes more movement than the part that is not lifted, which may cause pain and injury due to chafing of the skin. Therefore, as described above, the cut portion 1200 may reduce the lifted area and prevent skin chafing due to such lifting.

The fastening portion 2000 fastens the injury cover portion 1000 to the body 10. In one embodiment, the fastening portion 2000 includes a first fastening member 2100, a second fastening member 2200, and a connecting member 2300.

The first fastening member 2100 extends from the end 1001 of the injury cover portion 1000. The first fastening member 2100 may be flexible and/or elastic.

The second fastening member 2200 extends from the other end 1002 of the injury cover portion 1000. The second fastening member 2200 may be flexible and/or elastic.

For example, the first fastening member 2100 and the second fastening member 2200 may be made of natural or synthetic materials having flexibility and/or elasticity, such as leather, rubber, polyurethane, or acrylic resin. In another embodiment, the first fastening member 2100 and the second fastening member 2200 may have a structure in which different materials are combined. For example, the first fastening member 2100 and the second fastening member 2200 may be provided in a structure in which fabrics made of different materials overlap each other to form a plurality of layers. In one embodiment, the injury cover portion 1000, the first fastening member 2100, and the second fastening member 2200 may be provided as an integrated body having the same material and structure.

The connecting member 2300 detachably connects the end of the first fastening member 2100 and the end of the second fastening member 2200 to each other. In one embodiment, the connecting member 2300 may be provided in a zipper manner. Alternatively, the connecting member 2300 may be provided in various types of fastening structures that allow the end of the first fastening member 2100 and the end of the second fastening member 2200 to be attachable to each other and detachable from each other. For example, the connecting member 2300 may be provided in various structures, such as a Velcro or button fastening structure.

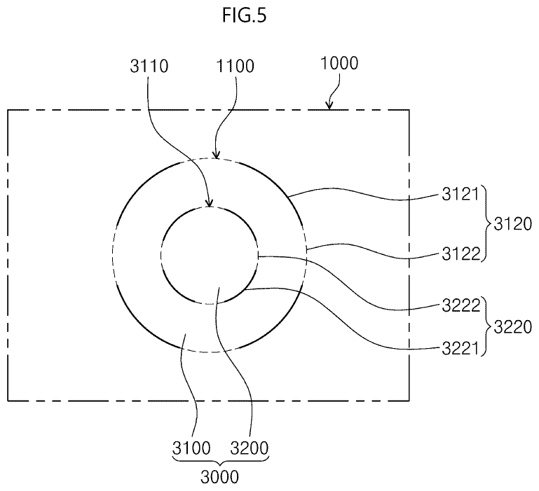

FIG. 4 is a front view illustrating an example of a fastening portion 2000 to which an extension member is connected. Referring to FIG. 4, the fastening portion 2000 may further include an extension member 2400. The extension member 2400 extends the length of the fastening portion 2000. In one embodiment, the end 2401 of the extension member 2400 is provided to be detachable from the end of the first fastening member 2100. The other end 2402 of the extension member 2400 is provided to be detachable from the end of the second fastening member 2200. A fastening configuration detachable from the configuration of the connecting member 2300 that is provided at the end of the first fastening member 2100 may be provided at the end 2401 of the extension member 2400. A fastening configuration detachable from the configuration of the connecting member 2300 that is provided at the end of the second fastening member 2200 may be provided at the other end 2402 of the extension member 2400. For example, when the connecting member 2300 is provided as a zipper structure, A configuration of a zipper detachable from the zipper configuration of the connecting member 2300 that is provided at the end of the first fastening member 2100 may be provided at the end 2401 of the extension member 2400. Another configuration of the zipper detachable from the zipper configuration of the connecting member 2300 that is provided at the end of the second fastening member 2200 may be provided at the other end 2402 of the extension member 2400. Alternatively, the connecting member 2300 and the extension member 2400 may be fastened to each other using a Velcro or button structure. The extension member 2400 allows the length of the injury contact prevention band 100 to be adjusted according to the circumference of the body part.

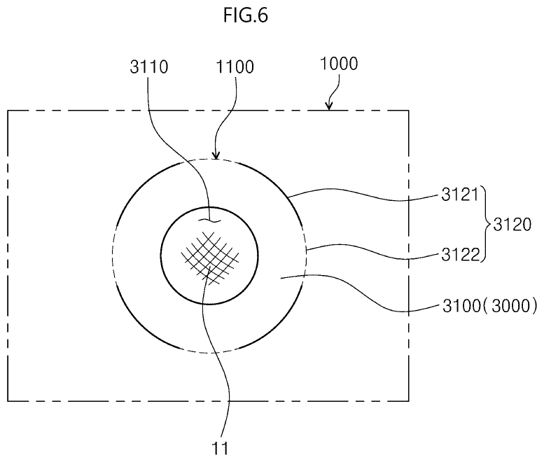

FIG. 5 is an enlarged front view of the injury opening hole 1100 illustrated in FIG. 1 with a hole cover portion 3000. FIG. 6 is a front view illustrating the hole cover portion 3000 and the injury opening hole 1100 in a state in which the second cover member 3200 illustrated in FIG. 1 is separated. The hole cover portion 3000 covers the injury opening hole 1100 and is connected to the injury cover portion 1000 in the injury opening hole 1100 so that the user may easily separate it from the injury opening hole 1100 by hand. If a plurality of injury opening holes 1100 are provided, a plurality of hole cover portions 3000 may be provided to correspond to the plurality of injury opening holes 1100 respectively.

In one embodiment, the outer perimeter surface of the hole cover portion 3000 fittingly engages, or accommodates, with the inner perimeter surface of the injury opening hole 1100. That is, the hole cover portion 3000 has a shape and size that may be inserted into the injury opening hole 1100 and completely cover the injury opening hole 1100. For example, when the injury opening hole 1100 has a circular or square shape, the hole cover portion 3000 correspondingly has a circular or square shape that engages the injury opening hole 1100. And, when the injury opening hole 1100 are provided in a slit shape, the hole cover portion 3000 is also provided in a shape corresponding to the injury opening hole 1100.

The hole cover portion 3000 includes a first cover member 3100 and a second cover member 3200. The first cover member 3100 may have a structure that surrounds the second cover member 3200. For example, the first cover member 3100 and the second cover member 3200 may have a concentric circle and/or ring structure with different diameters.

The outer perimeter surface of the first cover member 3100 may engage with the inner perimeter surface of the injury opening hole 1100. A first open hole 3110 penetrating between the inner surface and the outer surface, or cutting through the thickness, of the first cover member 3100 is located in the first cover member 3100.

In one embodiment, a first gap area 3120 may be positioned between the first cover member 3100 and the injury opening hole 1100 when the first cover member 3100 is inserted into the injury opening hole 1100 and connected to the injury opening hole 1100. The first gap area 3120 may include a first separation area 3121 and a first connection area 3122.

The first separation area 3121 is an area in which the hole cover portion 3000 and the injury cover portion 1000 are separated from each other in the first gap area 3120. The first connection area 3122 is an area where the first cover member 3100 and the injury cover portion 1000 are connected to each other in the first gap area 3120. A plurality of first separation areas 3121 and a plurality of first connection areas 3122 are provided alternately along the circumferential direction of the first gap area 3120. The length of the first connection area 3122 in the circumferential direction of the first gap area 3120 is shorter than the length of the first separation area 3121 in the circumferential direction of the first gap area 3120. A plurality of the first connection areas 3122 may be arranged to be spaced apart from each other at regular intervals. The first connection area 3122 is provided with a length in the circumferential direction of the first gap area 3120 that may be easily cut by hand, but may not be easily separated due to unintentional collision or contact. Accordingly, the length of the first connection area 3122 in the circumferential direction of the first gap area 3120 may vary depending on the material of the injury cover portion 1000. The length of the first connection area 3122 in the circumferential direction of the first gap area 3120 may be determined through experiment.

The second cover member 3200 covers the first open hole 3110. The outer perimeter surface of the second cover member 3200 engages with the inner perimeter surface of the first open hole 3110.

In one embodiment, a second gap area 3220 may be located between the second cover member 3200 and the first open hole 3110 when the second cover member 3200 is inserted into the first open hole 3110 and connected to the first cover member 3100. The second gap area 3220 may include a second separation area 3221 and a second connection area 3222.

The second separation area 3221 is an area in which the first cover member 3100 and the second cover member 3200 are separated from each other in the second gap area 3220. The second connection area 3222 is an area where the first cover member 3100 and the second cover member 3200 are connected to each other in the second gap area 3220. A plurality of second separation areas 3222 and a plurality of second connection areas 3222 are provided alternately along the circumferential direction of the second gap area 3220. The length of the second connection area 3222 in the circumferential direction of the second gap area 3220 is shorter than the length of the second separation area 3221 in the circumferential direction of the second gap area 3220. A plurality of second connection areas 3222 may be arranged to be spaced apart from each other at regular intervals. The second connection area 3222 has a length in the circumferential direction of the second gap area 3220 that may be easily cut by hand, but may not be easily separated due to unintentional collision or contact. Accordingly, the length of the second connection area 3222 in the circumferential direction of the second gap area 3220 may vary depending on the material of the injury cover portion 1000. The length of the second connection area 3222 in the circumferential direction of the second gap area 3220 may be determined through experiment.

In one embodiment, the direction in which each second connection area 3222 is located from the center of the hole cover portion 3000 may coincide with the direction in which each first connection area 3122 is located from the center of the hole cover portion 3000. Therefore, the force when tearing off the second cover member 3200 is transmitted directly to the first connection area 3122, so that compared to when the force is transmitted through the first separation area 3121, the stress concentrated in the first connection region 3012 is lowered. Accordingly, when the second cover member 3200 is torn off, the probability that the first cover member 3100 is torn off together may be reduced.

In addition, the length of the second connection area 3222 in the circumferential direction of the second gap area 3220 may be shorter than the length of the first connection area 3122 in the circumferential direction of the first gap area 3120. therefore, the probability that the first connection area 3122 is torn apart by a force that may separate the second connection area 3222 may be reduced.

In a state in which the hole cover portion 3000 is integrated with the injury cover portion 1000, the first separation area 3121, the second separation area 3221, the first connection area 3122, and the second connection area 3222 may be formed by a punching processing method. In contrast, the first separation area 3121, the second separation area 3221, the first connection area 3122, and the second connection area 3222 may be formed in the injury cover portion 1000 by various processing methods.

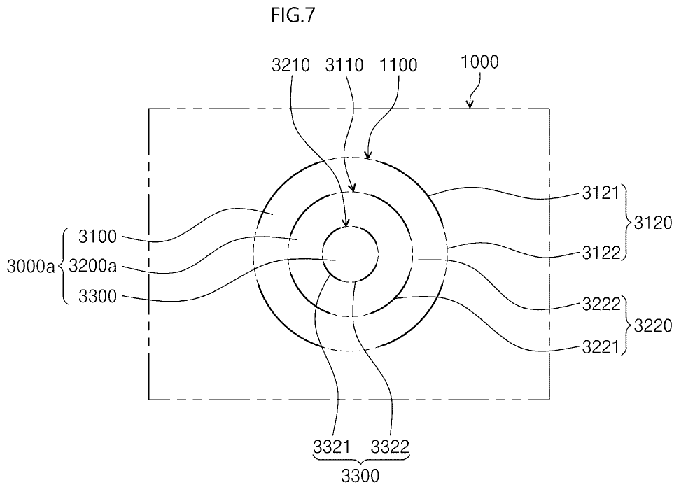

FIG. 7 is a front view illustrating a hole cover portion 3000a according to an example embodiment. Referring to FIG. 7, Unlike the hole cover portion 3000 of FIGS. 5 and 6 having a double structure, the hole cover portion 3000a may have a triple or more multi-layer structure. For example, the hole cover portion 3000a may further include a third cover member 3300. A second open hole 3210 penetrating between the inner surface and outer surface, or cutting through a thickness, of the second cover member 3200a is located in the second cover member 3200a. The third cover member 3300 covers the second open hole 3210. The outer perimeter surface of the third cover member 3300 fittingly engages with, or accommodates, the inner perimeter surface of the second open hole 3210.

In one embodiment, when the third cover member 3300 is inserted, pushed, or pressed into the second open hole 3210 and connected to the second cover member 3200, the third gap area 3300 may be located between the third cover member 3300 and the second open hole 3210. The third gap area may include a third separation area 3321 and a third connection area 3322. The above-described features between the first open hole 3110, the second cover member 3200, and the second gap area 3220 illustrated in FIGS. 5 and 6 may be applied identically or similarly between the second open hole 3210, the third cover member 3300 and the third gap area 3300.

If a hole of excessive size compared to the injury 11 is opened in the injury cover portion 1000, the area supporting the body weight of the injury cover portion 1000 may become narrow, thereby lowering the weight dispersion rate. If a hole of a smaller size than the injury 11 is opened in the injury cover portion 1000, a problem may occur in which the injury cover portion 1000 and the injury 11 come into contact. Since the hole cover portions 3000, 3000a may have a multiple structure, the injury contact prevention band 100 may utilize the hole of an appropriate size for the injuries of various sizes.

The features of the first gap area 3120 and the second gap area 3220 described with reference to FIG. 5 to 7 are applicable to the injury opening holes 1100 provided in various shapes as described above.

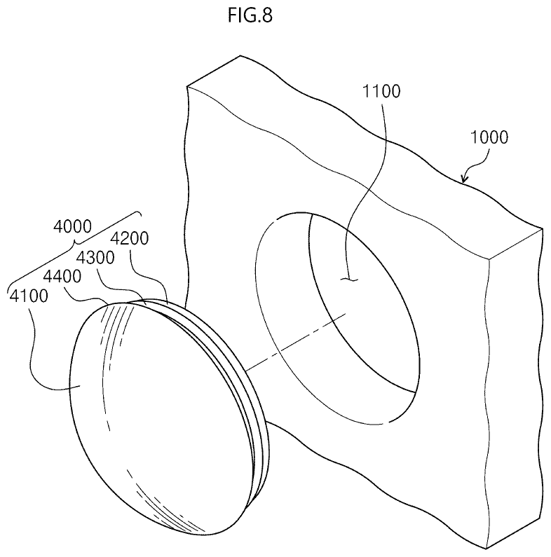

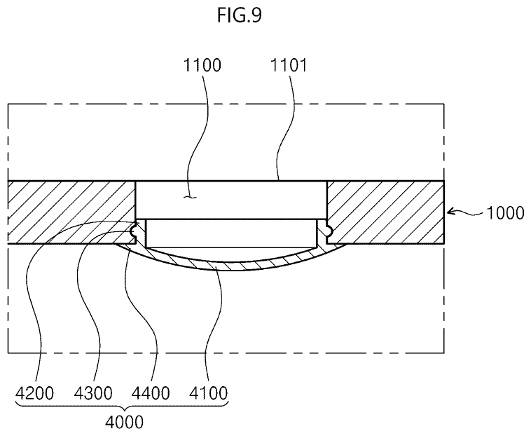

FIG. 8 is a perspective view illustrating an example of the hard cap portion 4000. FIG. 9 is a side cross-sectional view illustrating the hard cap portion 4000 of FIG. 8 inserted into the injury opening hole 1100. The injury contact prevention band 100 may further include a hard cap portion 4000. The hard cap portion 4000 is spaced apart from the inner opening 1101 in FIG. 3 of the injury opening hole 1100 and covers the outer opening 1102 in FIG. 3 of the injury opening hole 1100. The hard cap portion 4000 is detachably fastened to the injury opening hole 1100. The hard cap portion 4000 has hardness. For example, the hard cap portion 4000 may be made of hard plastic such as phenol resin, epoxy resin, or polyester resin, or hard metal such as titanium alloy. Alternatively, the hard cap portion 4000 may be made of various hard materials. According to an example embodiment, the hard cap portion 4000 includes a cover wall 4100, a side wall 4200, a fastening protrusion 4300, and a fringe portion 4400.

The cover wall 4100 covers the outer opening 1102 in FIG. 3 of the injury opening hole 1100 when the hard cap portion 4000 is inserted, pushed, or pressed into the injury opening hole 1100. The cover wall 4100 has a shape and size such that outer circumference thereof fittingly engages, or accommodates, the inner circumference of the injury opening hole 1100. For example, when the injury opening hole 1100 is provided in a circular, square, or slit shape, the cover wall 4100 may be provided in a circular, square, or slit shape accordingly, respectively. The cover wall 4100 may have a shape that is gently convex outward. Therefore, in response to the fact that the shape of the injury cover portion 1000 is generally convex outward when wrapping around the body 10, the patient wearing it may feel a more natural comfort.

The side wall 4200 protrudes inward from the inner surface of the cover wall 4100 when the hard cap portion 4000 is inserted, pushed, or pressed into the injury opening hole 1100. The side wall 4200 may have a cylindrical shape with an outer perimeter surface fittingly engaged with the inner perimeter surface of the injury opening hole 1100.

The protrusion length of the side wall 4200 from the cover wall 4100 may be smaller than the thickness of the outer perimeter surface of the injury opening hole 1100. In one embodiment, the protrusion length of the side wall 4200 from the cover wall 4100 is shorter than a length that the cover wall 4100 may protrude from the inner opening 1101 in FIG. 3 when the injury cover portion 1000 is pressed by the body weight of the patient wearing the injury contact prevention band 100. Therefore, even when the injury cover portion 1000 is pressed by the patient wearing it, the patient may feel little or unnoticeable foreign body sensation. The protrusion length of the side wall 4200 from the cover wall 4100 may be set through simulation or experiment and/or may depend on the type of injury or wound.

The fastening protrusion 4300 protrudes from the outer perimeter surface of the side wall 4200 in the direction in which the outer perimeter surface is facing. In one embodiment, the fastening protrusion 4300 may have a single closed curve shape along the outer circumferential direction of the side wall 4200. For example, when the injury opening hole 1100 is provided in a circular shape, the fastening protrusion 4300 may have a ring shape. In one embodiment, so that the hard cap portion 4000 may be more easily inserted into the injury opening hole 1100, the fastening protrusion 4300 may be inclined so that the protrusion length from the outer perimeter of the side wall 4200 gradually becomes longer toward the outside from the end facing inward to a predetermined position. With the fastening protrusion 4300, the hard cap portion 4000 may more firmly maintain the state of being inserted into the injury opening hole 1100.

The fringe portion 4400 extends from the outer perimeter of the cover wall 4100 along the direction in which the outer perimeter surface of the side wall 4200 is facing. In one embodiment, the fringe portion 4400 has an extended length longer than the protruding length of the fastening protrusion 4300. When the hard cap portion 4000 inserted into the injury opening hole 1100, the thickness from the outer surface of the injury cover portion 1000 of the fringe portion 4400 maybe gradually becomes thicker toward the outside from the outer perimeter thereof to the cover wall 4100. Additionally, the outer surface of the fringe portion 4400 may have a curved shape that extends in the curved direction of the outer surface of the cover wall 4100. Therefore, when the injury cover portion 1000 is pressed by the patient, the patient may move more flexibly and the patient feels less of a foreign body sensation. Accordingly, the fringe portion 4400 may prevent the hard cap portion 4000 from being pushed into the injury opening hole 1100.

The hard cap portion 4000 may more firmly prevent contact of the injury surrounded by the inner perimeter surface of the injury opening hole 1100 and reduce penetration of external foreign substances into the injury may be minimized.

When the hole cover portion 3000 has a multiple structure such as the first open hole 3110 described above, the hard cap portion 4000 may have various sizes corresponding to each hole.

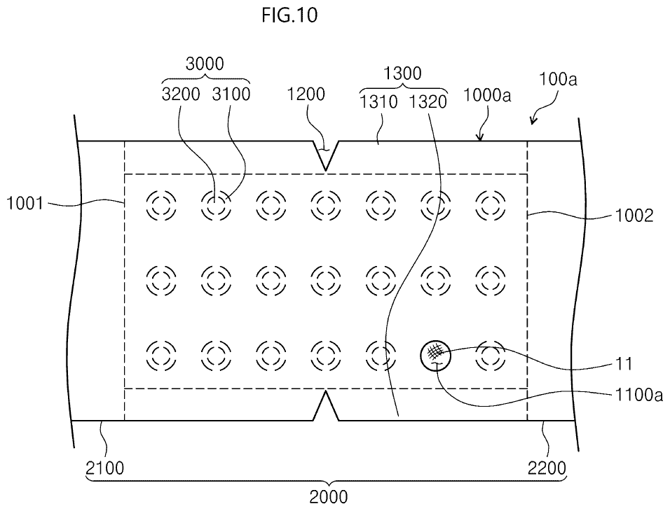

FIG. 10 is a partial front view illustrating an injury contact prevention band 100a according to an example embodiment. A plurality of injury opening holes 1100a may be arranged along at least one direction. For example, a plurality of injury opening holes 1100a may be arranged in a grid structure. Alternatively, the injury opening holes 1100a may be provided in various numbers and arranged along various directions. In this case, part or all of the injury opening holes 1100a may be opened as needed. A large number of the injury opening holes 1100a at various positions may be more easily positioned on the injury 11 at various positions. When a plurality of the injury opening holes 1100a exists, injury opening holes 1100a of different shapes may be formed in one the injury contact prevention band 100a. Additionally, when a plurality of the injury opening holes 1100a is available, a plurality of the hole cover portions 3000 may be positioned to correspond to each injury opening hole 1100a.

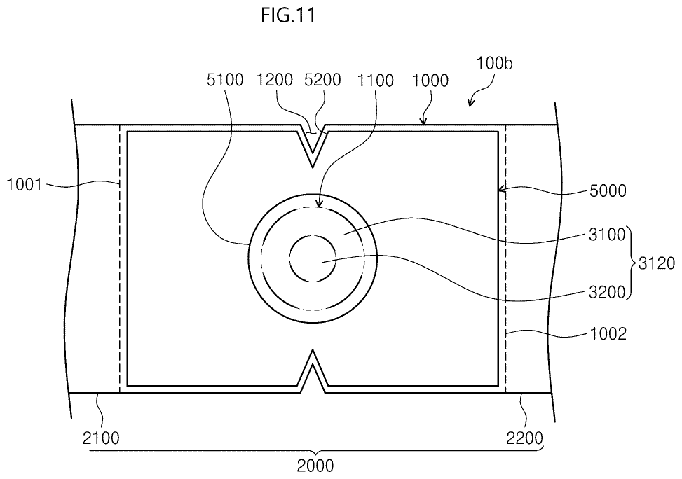

FIG. 11 is a partial front view illustrating an injury contact prevention band 100b according to an example embodiment. The injury contact prevention band 100b may further include a hard cover portion 5000.

The hard cover portion 5000 covers the outer surface of the injury cover portion 1000. The hard cover portion 5000 has hardness and ductility. The hard cover portion 5000 may be made of a metal material that has hardness and ductility. For example, the hard cover portion 5000 may be made of low carbon steel, copper, aluminum, or an alloy material containing these metal materials. the aluminum or the aluminum alloy may be the same material used for finger sprints.

An injury opening hole opening 5100 is located in the hard cover portion 5000. The injury opening hole opening 5100 cuts through the thickness of the injury cover portion 1000, or penetrates between the inner surface and outer surface of the injury cover portion 1000 to expose, reveal, show, or display the injury opening hole 1100 to the outside. In one embodiment, the injury opening hole opening 5100 has an inner circumference that surrounds the injury opening hole 1100.

The hard cover portion 5000 may be deformed according to the curvature of the body 10, so that the injury cover portion 1000 may be in close contact with the body as a whole to reduce areas of the inner surface of the injury cover portion 1000 lifted from the body 10. Therefore, the area of the injury cover portion 1000 where weight is distributed may expand, and chafing due to lifting of the injury cover portion 1000 may be prevented. In one embodiment, a cut groove 5200 is located in the hard cover portion 5000. The cut groove 5200 is indented from the outer perimeter of the hard cover portion 5000 toward the center of the hard cover portion 5000 in a shape opposite to the cut portion 1200 at a position opposite the cut portion 1200 to expose the cut portion 1200 to the outside. When the end area 1300 is deformed according to the curvature of the body 10 the cut groove 5200 may help preventing the area opposite to the end area 1300 of the hard cover portion 5000 from bending outward or inward so that foreign body sensation may be prevented when the injury contact prevention band is worn.

When a plurality of injury opening holes 1100a in FIG. 10 is available, the injury opening hole openings 5100 may be located at a position opposite to injury opening holes 1100a respectively, corresponding to the plurality of injury opening holes 1100a in FIG. 10.

When the hard cap portion 4000 is inserted, pushed, or pressed into the injury opening hole 1100, the injury opening hole opening 5100 may have an inner perimeter fittingly engaged with the outer perimeter of the fringe portion 4400, or have a size in which the inner perimeter of the injury opening hole opening 5100 is spaced apart from the branch portion 4400 and surrounds an area adjacent to the fringe portion 4400.

The injury contact prevention band 100, 100a, and 100b according to an example embodiment of the present disclosure may prevent contact with the injury 11 as the inner perimeter surface of the injury opening hole 1100 surrounds the injury 11. In addition, the injury contact prevention band according to an embodiment of the present disclosure is lighter and has a smaller volume than bedding such as a mattress for preventing injury contact, so the injury contact prevention band may be easily carried. In addition, the injury contact prevention band according to an example embodiment of the present disclosure may be fastened to various parts of the body 10, and may selectively open some of the plurality of injury opening holes 1100, so that the contact with the injury at the various parts of the body 10 may be prevented. In addition, the injury opening holes 1100 corresponding to the uninjured area are not opened unnecessarily, thereby lowering the penetration rate of foreign substances, and the hole cover portion 3000 supports the body weight so that the force that the uninjured area receives may be distributed.

While the present disclosure has been described with reference to embodiments thereof, it will be apparent to those of ordinary skill in the art that various changes and modifications may be made thereto without departing from the spirit and scope of the present disclosure as set forth in the following claims.

What is claimed is:

1. An injury contact prevention band comprising:
an injury cover portion configured to cover a part of a body including an injury and having a thickness through which an injury opening hole cuts to expose the injury;
a fastening portion configured to fasten the injury cover portion to the body; and
a hole cover portion configured to cover the injury opening hole, and to be separatable from the injury opening hole,
wherein the fastening portion includes:
a first fastening member extending from an end of the injury cover portion and having flexibility,
a second fastening member extending from another end of the injury cover portion and having flexibility, and
a connecting member detachably connecting an end of the first fastening member and an end of the second fastening member to each other, and
wherein the hole cover portion includes:
a first cover member in which an outer perimeter surface thereof fittingly engages with an inner perimeter surface of the injury opening hole and a first open hole is located, and
a second cover member covering the first open hole, and
wherein the outer perimeter surface thereof fittingly engages with an inner perimeter surface of the first open hole.

2. The injury contact prevention band of claim 1,
wherein a first gap area is located between the first cover member and the injury opening hole when the first cover member is inserted into the injury opening hole,
wherein the first gap area includes:
a first separation area where the first cover member and the injury cover portion are separated from each other, and
a first connection area where the first cover member and the injury cover portion are connected to each other,
wherein the first connection area has smaller length in the circumferential direction of the first gap area than length of the first separation area in the circumferential direction of the first gap area.

3. The injury contact prevention band of claim 2,
wherein a second gap area is located between the second cover member and the first open hole, when the second cover member is inserted into the first open hole,
the second gap area includes:
a second separation area where the first cover member and the second cover member are separated from each other, and
a second connection area where the first cover member and the second cover member are connected to each other,
wherein the second connection area has smaller length in the circumferential direction of the second gap area than length of the first connection area in the circumferential direction of the first gap area.

4. The injury contact prevention band of claim 1,
wherein the injury cover portion has a cut portion indented from an outer perimeter toward a central area thereof in an end area between an end and another end of an edge area thereof.

5. An injury contact prevention band comprising:
an injury cover portion configured to cover a part of a body including an injury and having a thickness through which an injury opening hole cuts to expose the injury;
a fastening portion configured to fasten the injury cover portion to the body; and
a hole cover portion configured to cover the injury opening hole, and to be separatable from the injury opening hole,
wherein the fastening portion includes:
a first fastening member extending from an end of the injury cover portion and having flexibility,
a second fastening member extending from another end of the injury cover portion and having flexibility, and
a connecting member detachably connecting an end of the first fastening member and an end of the second fastening member to each other, and
wherein the hole cover portion includes:
a first cover member in which an outer perimeter surface thereof fittingly engages with an inner perimeter surface of the injury opening hole and a first open hole is located, and
a second cover member covering the first open hole, and
wherein the outer perimeter surface thereof fittingly engages with an inner perimeter surface of the first open hole,
wherein a first gap area is located between the first cover member and the injury opening hole when the first cover member is inserted into the injury opening hole,
wherein the first gap area includes:
a first separation area where the first cover member and the injury cover portion are separated from each other, and
a first connection area where the first cover member and the injury cover portion are connected to each other,
wherein the first connection area has smaller length in the circumferential direction of the first gap area than length of the first separation area in the circumferential direction of the first gap area,
wherein a second gap area is located between the second cover member and the first open hole, when the second cover member is inserted into the first open hole,
the second gap area includes:
a second separation area where the first cover member and the second cover member are separated from each other, and
a second connection area where the first cover member and the second cover member are connected to each other,
wherein the second connection area has smaller length in the circumferential direction of the second gap area than length of the first connection area in the circumferential direction of the first gap area,
wherein the injury cover portion has a cut portion indented from an outer perimeter toward a central area thereof in an end area between an end and another end of an edge area thereof.
